(19) United States
(12) Patent Application Publication
(10) Pub. No.: US 2022/0229062 A1
(43) Pub. Date: Jul. 21, 2022

(54) METHOD FOR RAPID IMMUNOHISTOCHEMICAL DETECTION OF AN ANTIGEN FROM A BIOLOGICAL SAMPLE

(71) Applicants: Marc Key, (US); Diagnostic BioSystems, Pleasanton, CA (US)

(72) Inventors: Bipin Gupta, Pleasanton, CA (US); Marc Key, Ojai, CA (US)

(73) Assignees: Diagnostic BioSystems, Pleasanton, CA (US); Marc Key, Ojai, CA (US)

(21) Appl. No.: 17/516,829

(22) Filed: Nov. 2, 2021

Related U.S. Application Data
(60) Provisional application No. 63/139,921, filed on Jan. 21, 2021.

(51) Int. Cl. G01N 33/58; G01N 1/28; G01N 1/42

(52) U.S. Cl. CPC G01N 33/582; G01N 1/42; G01N 1/2813

(57) ABSTRACT

A method to produce a rapid immunohistochemical detection of an antigen from a biological sample is provided. Preferably, the method may be used for rapid immunohistochemical staining of a biological sample that allows completion of the staining process in less than ten minutes, such as during a cancer removal procedure. In some embodiments, the method may include the steps of: depositing a section of the biological sample on a slide; permeabilizing the section of the biological sample; incubating the section of the biological sample with a secondary antibody having a detectable label; removing unbound secondary antibody from the section of the biological sample; mounting the section of the biological sample; and detecting secondary antibody bound to the section of the biological sample.

### CROSS REFERENCE TO RELATED APPLICATIONS

This application claims priority to and the benefit of the filing date of U.S. Provisional Application No. 63/139,921, filed on Jan. 21, 2021, entitled “Rapid Stain for Tissue Proteins”, which is hereby incorporated by reference in its entirety.

### FIELD OF THE INVENTION

The present invention relates generally to the field of immunohistochemistry and more specifically, it relates to a rapid stain for tissue proteins to produce an immunohistochemical stain of antigens within a biological sample.

### BACKGROUND

Biological samples of cells and/or tissues are obtained for purposes of analyzing the biological constituents that comprise the sample. Sample analysis may be performed by methods of immunohistochemistry for analyzing protein components, or in situ hybridization for analyzing nucleic acid components. Prior to the analysis the sample must be prepared by appropriate methods. For example, a tissue biopsy is removed from a patient and processed by a standard histological method for embedding the biopsy into a solid block of paraffin. The tissue is embedded into a solid block to provide a firm surrounding matrix to facilitate sectioning. Briefly the tissue biopsy is surgically removed and placed into a fixative, such as formalin. Other fixatives may also be used but formalin in the most common. The formalin is typically used at about 10% in a buffered solution. Fixation time may be any time between 2-48 hours. A fixation time of about 8-24 hour is typical. The fixed tissue is then dehydrated through a series of alcohols and then the alcohol is removed and replaced with a paraffin solvent such as xylene. The alcohol is removed from the tissue by placing the tissue into successive baths containing increasing concentrations of xylene until the tissue is in 100% xylene. The xylene is then removed and replaced with paraffin by placing the tissue into successive baths with increasing concentrations of paraffin. The baths must be kept above the melting point of paraffin (about 60 C) to keep the solutions from solidifying. After the tissues are in 100% paraffin, they are formed into blocks and allowed to cool. As the cooling proceeds the blocks containing the tissues solidify. Once solidified the blocks containing the tissues are placed into a microtome that cuts thin sections of the tissues. Each thin section is about 4 u in thickness. The thin sections are applied to a microscope slide in preparation for immunohistochemical staining. The process so described is the most common method for preparing a tissue for microscopic examination. However, the process is time consuming generally requiring several hours to complete. Consequently, an alternative method may be employed in situations where short times are critical. In contrast to utilizing paraffin-embedded tissues, a shortened method for preparing a biopsy relies on the use of frozen tissues. The frozen tissue method has the following steps:

1. Fixation: Tissue is fixed to stop all metabolic activity and to preserve the molecular structure of the tissue. This may include chemical fixation, such as formalin, or fixation by freezing. The frozen tissue can be flash frozen in liquid nitrogen or a low temperature bath of isopentane and dry ice. The solid frozen tissue can then be embedded into a block and attached onto a holder frequently referred to as a chuck. The holder is typically a metal disc onto which the tissue is placed. An embedding medium may be used in order to attach the frozen tissue to the chuck. The tissue is now ready for sectioning on a cryostat. The cryostat is a device that can cut thin sections from the frozen tissue while maintaining a sub-zero temperature, such as −20 C. The tissue is typically sectioned at about 4-8 u to produce very thin tissue sections. The tissue sections are then placed onto a cold microscope slide where they become adherent. Typically, the microscope slides will have a charged surface that aids in tissue adherence. The microscope slides with adherent tissue are ready for pre-treatment in preparation for immunohistochemical staining.

2. Immunohistochemical Staining with Primary Antibody: The tissues are ready for immunohistochemical staining. For all immunohistochemical staining steps an appropriate buffer must be used. The typical buffer would be similar to physiological conditions in terms of molarity and pH. These conditions favor the binding of antibodies to antigens and enzyme-substrate reactions.

The first step in the staining process is to apply the primary antibody in a suitable buffer solution. The antibody is selected to bind to the antigen of interest, where the antigen of interest may be a protein that helps identify the particular tissue or tumor under investigation. If the primary antibody binds, then the antigen of interest is present, whereas if the primary antibody does not bind, then the antigen of interest is absent. A positive binding event will allow identification of the tumor cells. The primary antibody is applied to the tissue and incubated for a sufficient length of time to allow binding to occur.

The primary antibody is selected to bind to the antigen of interest. The primary antibody is typically generated by immunizing an animal with the antigen of interest. Commonly primary antibodies are produced in mice or in rabbits, although other animals can also be used. Typically, the antibodies are monoclonal antibodies, where the antibody is generated from a single clone of antibody-producing cells, or the antibodies may be polyclonal where the antibodies are produced from multiple different antibody-producing cells. A polyclonal antibody may be harvested from the serum of the immunized animal and in this state may be referred to as antiserum.

3. Immunohistochemical Staining with Tagged Secondary: The second step in this rapid immunohistochemistry process is to determine whether a binding event of primary antibody to antigen has occurred. In order to make this determination a secondary antibody is next applied. The secondary antibody has several unique features that allow it to be used in this respect. First the secondary antibody must be specific for the first primary antibody. For example, if the primary antibody has been produced in a mouse, then the secondary antibody must be anti-mouse immunoglobulin (Ig) specific. If the primary antibody has been produced in a rabbit then the secondary antibody must by anti-rabbit Ig specific. The second feature of the secondary antibody is that it must have a detectable tag such as an enzyme. Commonly used enzymes in immunohistochemistry include peroxidase and alkaline phosphatase.

4. Immunohistochemical Staining for Detection: After binding of the secondary antibody to the primary antibody the complex now contains a detectable label such as an enzyme. Common enzymes include alkaline phosphatase and peroxidase, most commonly peroxidase from horseradish peroxidase. There are numerous substrate/chromogen combinations that can be used with these enzymes that produce an insoluble colored reaction product that is deposited onto the microscope slide at the location where the primary antibody has bound. The presence of a colored reaction product is indicative of a positive test, meaning that the primary antibody has bound to its antigen. A common chromogen for use with alkaline phosphatase includes Fast Red which produces a red reaction product, and a common chromogen for use with peroxidase includes diaminobenzidine (DAB) which produces a brown reaction product at the site of an antigen-antibody reaction.

Once the secondary antibody has bound to the primary antibody, a detectable enzyme is now present. The next incubation step is to add a suitable substrate/chromogen mixture. The resultant enzyme-substrate reaction results in a chromogen being converted from a colorless form to a colored form. Furthermore, the chromogen is insoluble and deposits as a precipitate at the site of the enzyme. This colored reaction product is visible microscopically and is indicative of a positive reaction. After completion of the immunohistochemical stain, the tissue may be further stained with a counterstain. The counterstain will stain all the tissue elements whether or not they also have an immunohistochemical stain. The counterstain is useful for studying the overall structure and morphology of the tissue as a whole. Thus, the immunohistochemical stain provides a molecular stain and the counterstain provides a morphological stain. The counterstain would be chosen such that it would have a different color from the immunohistochemical stain so that it does not obscure the stain.

5. Preparing the Slides for Microscopic Examination: Having completed the immunohistochemical stain and the counterstain, the final steps include preparing the slides for microscopic examination. Preferred mounting procedures utilize resin-based mounting medium often referred to as permanent mounting medium. These mounting mediums contain resins and plastic dissolved in an organic solvent such as toluene or xylene. Because these mounting mediums are insoluble with water, all the water in the tissues must be removed prior to mounting. In a typical mounting procedure, the stained tissues are dehydrated in a series of alcohols beginning with a mixture of water and alcohol and gradually moving through a series of baths with decreasing water and increasing alcohol until the slides are in a bath of 100% alcohol. Next the alcohol is gradually removed and replaced with a solvent such as xylene or toluene by moving the slides through a series of baths beginning first with a bath of alcohol and solvent and then moving through a series of baths with decreasing alcohol and increasing solvent until the slides are in 100% solvent. Finally at this stage the slides are ready for mounting. The slides are removed from the last solvent bath, and a drop of mounting medium is applied. Next a glass coverslip is overlaid the mounting medium. The mounting medium dries and glues the coverslip over the tissue. The tissues are now ready for microscopic examination.

Even though the frozen tissue method of preparing a tissue is much faster than the paraffin-embedded method, neither method is fast enough to meet the requirements for fast turn around time from sample collection to results. Therefore, a need exists for novel methods to be used for providing rapid immunohistochemical detection of an antigen from a biological sample.

### BRIEF SUMMARY OF THE INVENTION

A method to produce a rapid immunohistochemical detection of an antigen from a biological sample is provided. Preferably, the method may be used for rapid immunohistochemical staining of a biological sample that allows completion of the staining process in less than ten minutes. Such rapid staining is essential in certain diagnostic settings. For example, in diagnosis of melanoma the patient may be required to remain in surgery until the staining and diagnosis is completed.

In some embodiments, the method may include the steps of: depositing a section of the biological sample on a slide; permeabilizing the section of the biological sample; incubating the section of the biological sample with a secondary antibody having a detectable label; removing unbound secondary antibody from the section of the biological sample; mounting the section of the biological sample; and detecting secondary antibody bound to the section of the biological sample.

In further embodiments, the method may be used during a cancer removal procedure and the method may include the steps of: depositing a section of the biological sample on a slide, in which the section of the biological sample is produced by freezing the biological sample and then sectioning the biological sample; permeabilizing the section of the biological sample; incubating the section of the biological sample with a secondary antibody having a detectable label, in which the secondary antibody is specific to a Fc portion of a primary antibody also applied to the section of the biological sample; removing unbound secondary antibody from the section of the biological sample; mounting the section of the biological sample, in which the step of mounting the section of the biological sample includes applying a mixture of an alcohol and an alcohol and water soluble polymer to the section of the biological sample; and detecting secondary antibody bound to the section of the biological sample.

It is an object of the present invention to produce a method for rapid immunohistochemical stain of antigens within a biological sample.

Another object is to provide a rapid stain for tissue proteins that uses a method to permeabilize the tissue.

Another object is to provide a rapid stain for tissue proteins that utilizes an Fc-specific enzyme conjugate to detect and bind to the primary antibody.

Another object is to provide a rapid stain for tissue proteins that utilizes a mixture of a primary antibody and a secondary antibody where the secondary antibody is an Fc-specific enzyme conjugate.

Another object is to provide a rapid stain for tissue proteins that utilizes method for rapid dehydration of the immunohistochemically stained biological sample.

Another object is to provide a rapid stain for tissue proteins that utilizes a method and reagent to permanently seal the immunohistochemically stained biological sample.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a block diagram of an example of a method to produce a rapid immunohistochemical detection of an antigen from a biological sample according to various embodiments described herein.

FIG. 2 illustrates a block diagram of an example of a method to rapidly permeabilize a biological sample according to various embodiments described herein.

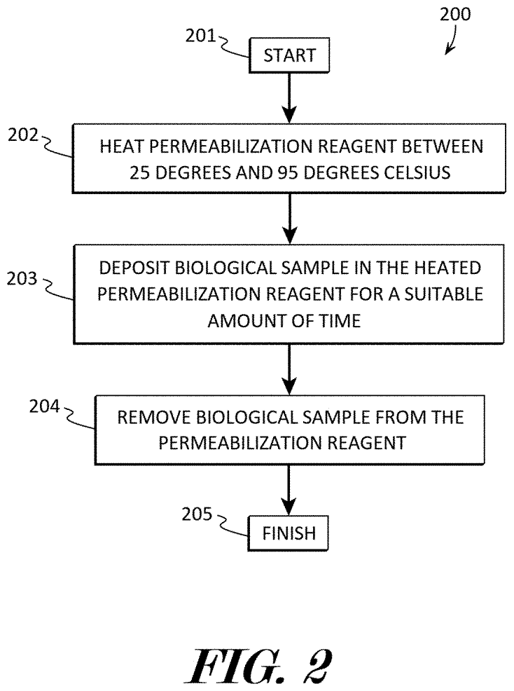

FIG. 3 shows a block diagram of an example of a method to rapidly bind an antigen of a biological sample with a labeled secondary antibody according to various embodiments described herein.

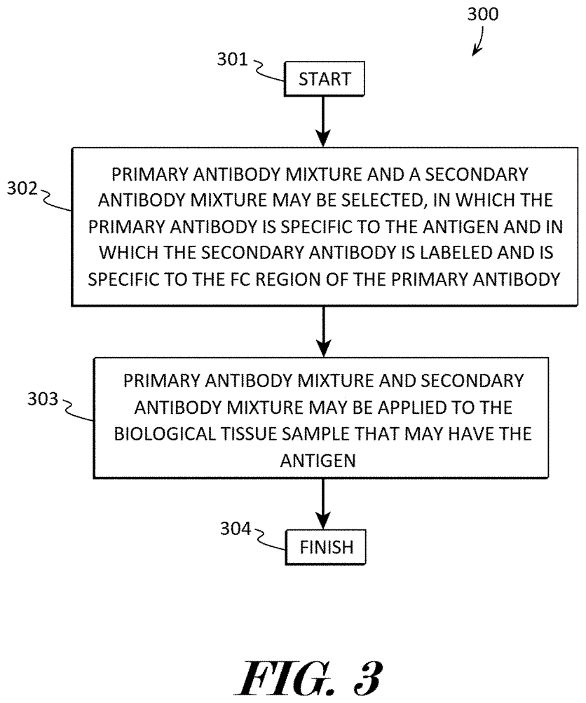

FIG. 4 depicts a block diagram of an example of a method of rapidly dehydrating a biological sample according to various embodiments described herein.

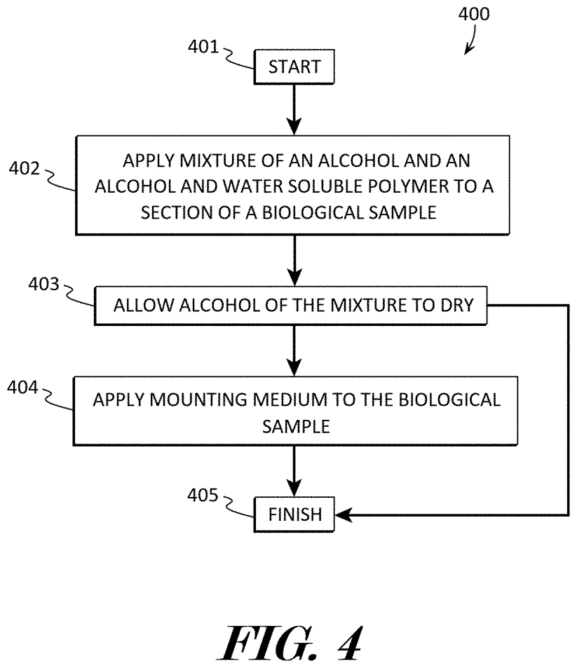

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

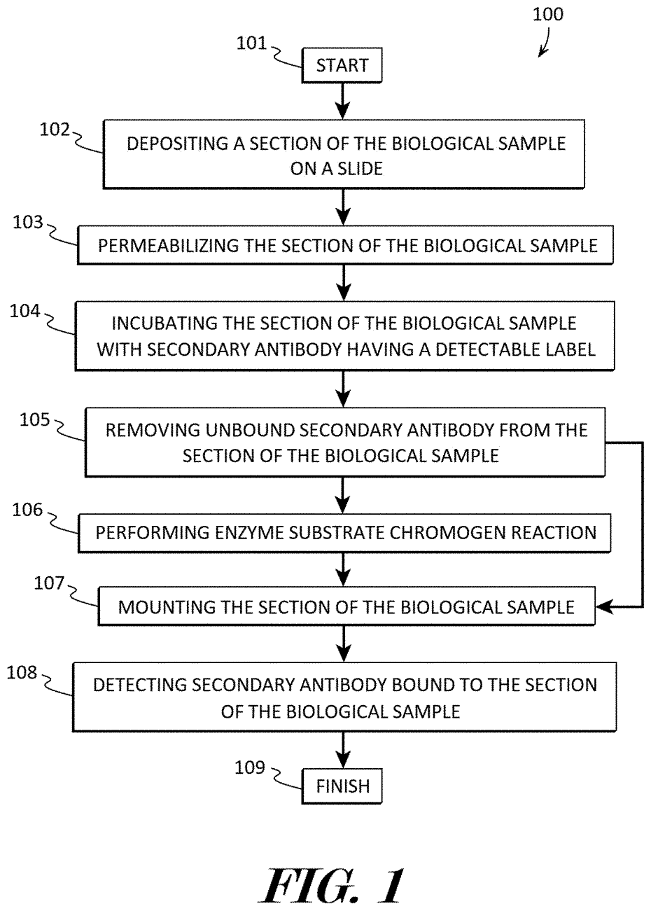

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new method for rapid immunohistochemical detection of an antigen from a biological sample is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments. FIG. 1 illustrates an example of a method for rapid immunohistochemical detection of an antigen from a biological sample (“the method”) 100 according to various embodiments. In some embodiments, the method 100 may be used for the rapid immunohistochemical staining of a biological sample that allows completion of the staining or detection process in less than ten minutes. Such rapid antigen detection is essential in certain diagnostic settings. For example, in diagnosis and removal of melanoma the patient may be required to remain in surgery until the detection and diagnosis is completed. In preferred embodiments, the method 100 may be a method for rapid immunohistochemical detection of an antigen from a biological sample during a cancer removal procedure, such as during Moh's micrographic surgery for skin cancer removal.

In some embodiments, the method 100 may start 101 and a section of the biological sample may be deposited on a slide is step 102. Preferably the biological sample may have been or may be fixed to stop all metabolic activity and to preserve the molecular structure of the tissue of the biological sample.

In some embodiments of the method 100, the section of the biological sample may be produced by embedding the biological sample in paraffin or other suitable embedding medium that may provide a firm surrounding matrix to facilitate sectioning and then the embedded biological sample may be sectioned. During conventional cancer removal procedures, such as Moh's micrographic surgery, the tissue is prepared and embedded into a paraffin block by standard histological methods. Briefly the tissue biopsy is surgically removed and placed into a fixative, such as formalin. Other fixatives may also be used but formalin in the most common. The formalin is typically used at about 10% in a buffered solution. Fixation time may be any time between 2-48 hours. A fixation time of about 8-24 hour is typical. The fixed tissue is then dehydrated through a series of alcohols and then the alcohol is removed and replaced with a paraffin solvent such as xylene. The alcohol is removed from the tissue by placing the tissue into successive baths containing increasing concentrations of xylene until the tissue is in 100% xylene. The xylene is then removed and replaced with paraffin by placing the tissue into successive baths with increasing concentrations of paraffin. The baths must be kept above the melting point of paraffin (about 60 degrees Celsius) to keep the solutions from solidifying. After the tissues are in 100% paraffin, they are formed into blocks and allowed to cool. As the cooling proceeds the blocks containing the tissues solidify. Once solidified the blocks containing the tissues are placed into a microtome that cuts thin sections of the tissues. Each thin section is about 4 micrometers in thickness. The thin sections are applied to a microscope slide in preparation for immunohistochemical staining.

In preferred embodiments of the method 100, the section of the biological sample may be produced by freezing the biological sample and then sectioning the biological sample. For rapid immunohistochemistry it is preferable to freeze the tissue rather than embed the tissue in paraffin because the time required to process a frozen tissue is significantly shorter than the time required to embed a tissue in paraffin. The frozen tissue can be flash frozen in liquid nitrogen or a low temperature bath of isopentane and dry ice. The solid frozen tissue can then be embedded into a block and attached onto a holder frequently referred to as a chuck. The holder is typically a metal disc onto which the tissue is placed. An embedding medium may be used in order to attach the frozen tissue to the chuck. The tissue is now ready for sectioning on a cryostat. The cryostat is a device that can cut thin sections from the frozen tissue while maintaining a sub-zero temperature, such as −20 C. The tissue is typically sectioned at about 4-8 micrometers to produce very thin tissue sections. The tissue sections are then place onto a cold microscope slide where they become adherent. Typically, the microscope slides will have a charged surface that aids in tissue adherence. The microscope slides with adherent tissue are ready for pre-treatment in preparation for immunohistochemical staining.

In step 103 the section of the biological sample may be permeabilized. In preferred embodiments, the microscope slide with the attached tissue is brought to room temperature, at which time the tissue melts and attaches to the microscope slide. Based on the molecular structure of the tissue and the specific antigens under investigation, the antigens may be hidden or otherwise inaccessible to the primary antibody. Therefore, it has been found to be useful to first permeabilize the tissue prior to staining. Although the exact mechanism by which the permeabilization reagent exposes hidden antigens is unknown it is thought to act by changing the molecular structure of the tissue and antigens. The detergent contained in the permeabilization reagent may destroy or dissolve lipid layers and other hydrophobic molecules thereby allowing the hydrophilic antibodies to more easily penetrate the tissues. Furthermore, chelating agents may be useful in helping to stabilize proteins.

In some embodiments, permeabilization procedures may be performed by submerging slides with attached tissues into the permeabilization reagent that is at room temperature, such as between 17 and 20 degrees Celsius, and incubated for a sufficient length of time that the tissues are permeabilized. The length of time necessary for permeabilization varies depending on the temperature of the incubation mixture.

In preferred embodiments of step 103, the section of the biological sample may be permeabilized via a method to rapidly permeabilize a biological sample (“the method”) 200. In some embodiments, the method 200 may start 201 and a permeabilization reagent may be heated to between 25 degrees and 95 degrees Celsius in step 202. In preferred embodiments, the permeabilization reagent may be heated to approximately 90 degrees Celsius. Next in step 203, the biological sample may be deposited in the heated permeabilization reagent for a suitable amount of time, such as between 10 seconds and 10 minutes. In preferred embodiments, the permeabilization reagent may be heated to approximately 90 degrees Celsius, and the biological sample may be deposited in the heated permeabilization reagent for between approximately 30 seconds to 90 seconds, and more preferably for approximately one minute. Furthermore, because of the short incubation time, the entire permeabilization step can be conducted at room temperature with only slight cooling of the permeabilization reagent. Next in step 204, the biological sample may be removed from the permeabilization reagent. After step 204, the method 200 may finish 205.

Permeabilization in step 103 may also be conducted at different temperatures, but the incubation times may have to be increased to achieve sufficient permeabilization. For example, sufficient permeabilization will occur even under conventional room temperature incubation conditions if the incubation time is extended to 60 minutes.

For rapid immunohistochemistry the permeabilization reagent may be pre-heated in step 202, such as in a microwave for about one minute, in order to achieve a temperature of about 90 degrees Celsius. Pre-heating can take place while the microscope slides are being sectioned, thus pre-heating does not add any time to the overall rapid immunohistochemistry staining process. Once the slides are placed into the heated permeabilization reagent in step 203, they are incubated for about one minute in a room temperature environment and then removed from the permeabilization reagent in step 204. After removal from the permeabilization reagent, the slides having the biological samples may be transferred to a buffer bath that is suitable for immunohistochemistry such as a Tris or Phosphate buffer at about pH 7.2.

Following permeabilization the tissues are ready for immunohistochemical staining. For all immunohistochemical staining steps an appropriate buffer must be used. The typical buffer would be similar to physiological conditions in terms of molarity and pH. These conditions favor the binding of antibodies to antigens and enzyme-substrate reactions.

In step 104 the biological sample may be incubated with a secondary antibody having a detectable label to determine whether a binding event of a primary antibody to an antigen of interest has occurred. A secondary antibody used in step 104 preferably may have several unique features that allow it to be used in this respect. First, the secondary antibody must be specific for a primary antibody applied to the biological sample that is specific to the antigen of interest that may be in the biological sample. For example, if the primary antibody has been produced in a mouse, then the secondary antibody must be anti-mouse immunoglobulin (Ig) specific. If the primary antibody has been produced in a rabbit, then the secondary antibody must by anti-rabbit Ig specific. The second feature of the secondary antibody is that it must have a detectable label or tag, such as an enzyme molecule, a polymer having two or more enzyme molecules bound to it, a fluorescent tag, etc. Commonly used enzymes in immunohistochemistry include peroxidase and alkaline phosphatase. Furthermore, enzymes may be directly attached to the secondary antibody or attached via a polymer backbone. The advantage of using a polymer backbone is that multiple same and/or different enzyme molecules and multiple same and/or different secondary antibodies can be contained within a polymerized molecular structure, thus providing increased sensitivity to the detection method. Antibody polymers can be produced by conjugating multiple antibody molecules to a polymer backbone. Additionally, multiple enzyme molecules can be conjugated to the antibody-polymer structure. In preferred embodiments, the method 100 may use polymers of antibody and two or more enzyme molecules in the performance of step 104 because they provide greater sensitivity compared to antibodies having a single enzyme molecule.

In preferred embodiments, step 104 may utilize a secondary antibody that is Fc-specific to a primary antibody also applied to the biological sample. Fc-specific antibodies have unique specificity in that they bind to only a certain region of a primary antibody and this region is termed the Fc portion. The Fc portion of an antibody is not involved in the binding of an antibody to its antigen. The antigen combining regions are termed Fab. Therefore, at one end of the antibody molecule are the Fab regions and on the other end of the antibody molecule are the Fc regions. A secondary antibody that is not Fc-specific will bind to all regions of the primary antibody including the Fab and Fc regions.

In conventional immunohistochemistry the primary antibody is incubated with the tissue for a sufficient length of time in order for the antibody to bind to its antigen. After a sufficient length of time has passed the tissue is rinsed such that all unbound primary antibodies are removed. Any bound primary antibodies will remain attached to the tissue. Next a secondary antibody is applied and incubated for a sufficient length of time that it will bind to its primary antibody, if present. However, in the present invention the primary antibody and the secondary antibody can be incubated together at the same time and/or the secondary antibody can be added to the slide at any point after the primary antibody without the requirement to first rinse off unbound primary antibody. The incubation of the primary antibody and secondary antibody can be performed in a variety of ways. The primary antibody and secondary antibody can be premixed, and then applied to the tissue as a single reagent. The primary antibody can be applied to the tissue and allowed to react for a short period of time, and then without rinsing the secondary antibody can be directly added to the incubation mixture. And finally, the primary antibody can be incubated for a short period of time, the excess reagent drained from the slide without rinsing, and then the secondary antibody added to the remaining incubation mixture.

For example, in some embodiments of step 104, the section of the biological sample may be incubated with a secondary antibody after incubating the section of the biological sample with a primary antibody that is specific to the antigen in which the secondary antibody is specific to the Fc portion of the primary antibody. As another example, in some embodiments of step 104, the section of the biological sample may be concurrently incubated with the secondary antibody and with a primary antibody that is specific to the antigen in which the secondary antibody is specific to the Fc portion of the primary antibody. Because of these unique characteristics the time required to stain, fluorescently tag, or otherwise label an antigen with a detectable label the tissue can be dramatically reduced by the method 100.

To better understand this reaction and the benefits of using an Fc-specific secondary antibody; let us first consider a standard immunohistochemistry staining method with a conventional anti-Ig secondary antibody. Because the secondary antibody has the capability of binding to the Fab region of the primary antibody, it cannot be incubated simultaneously with the primary antibody. If this were attempted the secondary antibody would bind to the Fab region of the primary antibody before the primary antibody had bound to its antigen. Such binding to the Fab region of the primary antibody would inhibit its binding to its antigen. In order for this system to work, the primary antibody must bind first to its antigen and this reaction must be complete prior to application of the secondary antibody. Furthermore, any excess unbound primary antibody must be rinsed off of the slide before the secondary antibody can be applied.

However, in the case of the method 100 of the present invention, this limitation may be overcome by selecting a secondary antibody that is Fc-specific. In this case the primary antibody and secondary antibody can be incubated together simultaneously, because the secondary antibody does not bind to the Fab regions and thus does not inhibit the binding of the primary antibody to its antigen. Although the secondary antibody will bind to the Fc region of the primary antibody during this incubation, this binding does not interfere with the binding of the primary antibody to its antigen. Therefore, in preferred embodiments, the method 100 may use an Fc-specific secondary antibody in step 104 so as to allow two separate conventional incubation steps to be performed as a single incubation step.

In preferred embodiments of step 104, section of the biological sample may be incubated with a secondary antibody having a detectable label using a method to rapidly bind an antigen of a biological sample with a labeled secondary antibody (“the method”) 300. In some embodiments, the method 300 may start 301 and a primary antibody mixture and a secondary antibody mixture may be selected, in which the primary antibody is specific to the antigen and in which the secondary antibody is labeled and is specific to the Fc region of the primary antibody, in step 302. Next in step 303 the primary antibody mixture and secondary antibody mixture may be applied to the biological tissue sample that may have the antigen. In some embodiments of step 303, the primary antibody mixture may first be applied to the biological sample and then the secondary antibody mixture may be applied to the biological sample while the primary antibody mixture is still in contact with the biological sample. In further embodiments of step 303, the secondary antibody mixture may first be applied to the biological sample and then the primary antibody mixture may be applied to the biological sample while the secondary antibody mixture is still in contact with the biological sample. In still further embodiments of step 303, the primary antibody mixture and the secondary antibody mixture may be simultaneously applied to the biological sample, such as by premixing the primary and secondary antibody mixtures before application. Once both the primary antibody mixture and the secondary antibody are applied to the biological sample, the method 300 may finish 304.

In step 105, unbound secondary antibody may be removed from the section of the biological sample. After a sufficient length of time has passed in step 104, such as between approximately 1 minute to 20 minutes, the tissue of the biological sample is rinsed with buffer or other suitable rinsing medium such that all unbound secondary antibodies are removed. By removing the unbound secondary antibodies, the unbound primary antibodies may also be removed. After step 105, the method 100 may proceed to step 106 or to step 107.

If the secondary antibody used in steps 104 and 105 was an enzyme labeled antibody, the method 100 may proceed to optional step 106 in which an enzyme substrate chromogen reaction may be performed by adding a suitable substrate/chromogen mixture biological sample. The resultant enzyme-substrate reaction results in a chromogen being converted from a colorless form to a colored form. Furthermore, the chromogen is insoluble and deposits as a precipitate at the site of the enzyme. This colored reaction product is visible microscopically and is indicative of a positive reaction. After completion of the immunohistochemical stain, the tissue may be further stained with a counterstain. The counterstain will stain all the tissue elements whether or not they also have an immunohistochemical stain. The counterstain is useful for studying the overall structure and morphology of the tissue as a whole. Thus, the immunohistochemical stain provides a molecular stain and the counterstain provides a morphological stain. The counterstain may be chosen such that it would have a different color from the immunohistochemical stain so that it does not obscure the stain.

There are many counterstains that can be applied to the tissue. The counterstain must have a color distinct from the immunohistochemical stain. In the case of DAB (brown) or Fast Red (Red), a blue counterstain would be appropriate. Hematoxylin provides a blue counterstain and is the most widely used counterstain in immunohistochemistry.

In step 107, the section of the biological sample may be mounted so as to be prepared for microscopic examination. Preferred mounting procedures utilize resin-based mounting medium often referred to as permanent mounting medium. These mounting mediums contain resins and plastic dissolved in an organic solvent, such as toluene or xylene. Because these mounting mediums are insoluble with water, all the water in the tissues must be removed prior to mounting. In a typical mounting procedure, and in some embodiments of step 106, the stained or fluorescently labeled tissues are dehydrated in a series of alcohols beginning with a mixture of water and alcohol and gradually moving through a series of baths with decreasing water and increasing alcohol until the slides are in a bath of 100% alcohol. Next the alcohol is gradually removed and replaced with a solvent such as xylene or toluene by moving the slide through a series of baths beginning first with a bath of alcohol and solvent and then moving through a series of baths with decreasing alcohol and increasing solvent until the slides are in 100% solvent. Finally at this stage the slides are ready for mounting. The slides are removed from the last solvent bath, and a drop of mounting medium is applied. Next a glass coverslip is overlaid the mounting medium. The mounting medium dries and glues the coverslip over the tissue. The tissues are now ready for microscopic examination using this conventional mounting procedure. However, this conventional mounting procedure is time intensive and can take upwards of 20 minutes to complete.

In preferred embodiments, step 107 may be performed using a method of rapidly dehydrating a biological sample (“the method”) 400. The method 400 may be used as a rapid and simplified method to dehydrate the tissues to prepare the biological sample for mounting after unbound secondary antibody is removed from the section of the biological sample. The method 400 may employ a mixture of an alcohol and an alcohol soluble polymer or plastic, such as 10% polyvinyl in ethanol. Additionally, the alcohol soluble polymer must also be soluble in water. Example alcohol and water soluble polymers include: Polymers of vinyl, such as polyvinyl alcohol, polyvinyl acetate, polyvinyl pyrrolidone, and polyvinyl butyral; Polymers of ethylene, such as polyethylene glycol; and Polymers of cellulose such as, ethyl cellulose, propyl cellulose, hydroxypropyl cellulose, hydroxymethyl cellulose, and hydroxypropyl methylcellulose.

In some embodiments, the method 400 may start 401 and a mixture of an alcohol and an alcohol and water soluble polymer, the mixture comprising an alcohol and plastic dehydrating reagent, may be applied to a section of a biological sample in step 402. Optionally, a mixture of the alcohol and plastic is prepared as needed for dehydration. In preferred embodiments, the alcohol and plastic dehydrating reagent is applied to the slide holding the biological sample such that any remaining water is rapidly displaced by the dehydrating reagent (generally by applying an excess of the alcohol and plastic dehydrating reagent). Because the plastic is also water soluble, it will not precipitate when exposed to the remaining water on the slide. In further preferred embodiments, and when used in the performance of a method for rapid immunohistochemical detection of an antigen from a biological sample 100, step 402 may be performed after counterstained slides are rinsed with water to remove excess counterstain, unbound antibody, etc.

In step 403, the alcohol of the alcohol and plastic dehydrating reagent is allowed to dry, such as by air drying. The air drying process takes approximately 10-20 seconds to complete because the alcohol dries very rapidly. Furthermore, the slide dries with a thin layer of the plastic embedded into the tissue and overlaying the tissue. The plastic provides structure and support to the tissue as it dries and prevents drying artifacts, such as tissue shrinkage or cracking that would occur in the absence of the plastic. Furthermore, the plastic layer can act as a barrier between the tissue and the mounting medium. This can act to protect chromogens from fading when in direct contact with the mounting medium. After step 403, the method 400 may finish 405 or the method 400 may proceed to optional step 404 so that the method 400 may function as a method of rapidly dehydrating and mounting a biological sample.

In optional step 404, mounting medium may be applied to the biological sample. In preferred embodiments, after the alcohol of step 403 applied to the biological sample slide is dried a drop of resin-based mounting medium, often referred to as permanent mounting medium, may be added on the biological sample and then overlaid with a glass coverslip. The entire process of the method 400, including optional step 404 that preferably uses a resin-based mounting medium, takes less than approximately 30 seconds. In further embodiments, step 404 may be performed using an aqueous mounting medium that is composed of one or more water-soluble plastics and polymers. This type of mounting medium can be added directly onto a wet microscope slide without the requirement for first dehydrating the slide. Although aqueous mounting is convenient, the optical resolution for aqueous mounting media are considered inferior to permanent mounting media. Thus, for best resolution resin-based (permanent) mounting media are preferred. After step 404, the method 400 may finish 405.

After step 107, the method 100 may continue to step 108 of detecting secondary antibody bound to the section of the biological sample, preferably via microscopic examination, which may be the final step in the immunohistochemistry detection process and the method 100 may finish 109. This step of detecting secondary antibody bound to the section of the biological sample is the same whether a conventional immunohistochemistry method or whether a rapid immunohistochemistry method 100 is used. However, the rapid immunohistochemistry method 100 is uniquely applicable to certain situations where rapid diagnosis is required. One such application is Moh's micrographic surgery, for example in the examination of skin cancers. This method requires excision of the suspected lesion, staining, and microscopic examination. In this examination the primary goal is to examine the surgical margins for the presence or absence of tumor cells. If tumor cells are present at the surgical margin, then the surgeon will remove additional tissue until the surgical margins are clear of tumor cells. Thus, the patient must remain for long periods of time while the tissues are stained and inspected. Typical stains used for this procedure include Hematoxylin and eosin (H&E) because they provide rapid results. However, H&E only provide morphological information and do not provide molecular information. Furthermore, a few tumor cells present near the surgical margins could be missed by this method of staining. Immunohistochemical staining could improve on this sensitivity but is rarely used because the long times associated with producing a stain. Thus, immunohistochemical stains are considered incompatible with this method. As can be seen by this brief description there is a need for a rapid immunohistochemical stain which could provide results in approximately the same time frame as a conventional H&E stain. The method 100 of the present invention is able to achieve this goal.

In step 108, after mounting the stained slides are ready for microscopic review. When viewed microscopically the tissues will typically show an abundance of counterstained cells, blue in the case of Hematoxylin. The microscopist will be able to discern certain morphological features such as tissue type and the presence or absence of tumor material. However, toward the margins of the tumor the identification of individual tumor cells becomes more difficult as they may be obscured by the more abundant normal cells. It is critical to identify all tumor cells at the margins of the surgical incision. If tumor cells are identified up to the surgical margins it is likely that some tumor cells remain in the body and further surgery is indicated.

The use of immunohistochemical staining methods provides increased sensitivity for detecting isolated tumor cells, particularly at the surgical margins. These isolated tumor cells will stain according to the chromogen used (typically brown or red) and are easily identified, even among an abundance of normal cells. Thus, the identification of colored reaction product is indicative of a positively-staining tumor cell. Despite this apparent improvement in sensitivity, immunohistochemistry is rarely used because the length of time required to process slides by immunohistochemistry is incompatible with the rapid staining requirements of the Moh's procedure.

Connections of Main Elements and Sub-Elements of Invention

The IHC staining method 100 has been developed specifically for rapid immunohistochemical staining. As such each of the steps has been optimized for speed. Although some of the steps that have been incorporated into this staining method utilize conventional histochemical or immunohistochemical techniques, many of the steps have been modified to accomplish staining within a 10 minute time frame. These steps include:

Step 102—depositing a section of the biological sample on a slide. In preferred embodiments, step 102 may comprise preparation of a biological sample by flash freezing and cutting a thin section of sample from the frozen block and affixing the sections onto a microscope slide.

Step 103—permeabilizing the section of the biological sample. In preferred embodiments, step 103 may comprise permeabilizing the tissue sample to expose hidden antigens via a permeabilization reagent that is heated to between approximately 25 degrees and 95 degrees Celsius. In further preferred embodiments, step 103 may comprise permeabilizing the tissue sample to expose hidden antigens via a permeabilization reagent that is heated to approximately 90 degrees Celsius for approximately one minute.

Step 104—incubating the section of the biological tissue sample with secondary antibody having a detectable label. In preferred embodiments, step 104 may comprise one or more of: staining the tissue with a primary antibody; staining or labeling the tissue with a secondary antibody either simultaneously with the primary antibody or sequentially without rinsing the primary antibody; staining or labeling with a secondary antibody that is Fc-specific with respect to the primary antibody; staining or labeling with a secondary antibody that is a polymer; staining or labeling with a secondary antibody that has a detectable enzyme attached; staining or labeling with a secondary antibody that has a fluorescent tag attached; and staining or labeling with a substrate/chromogen that is specific to the detectable enzyme, thereby producing an insoluble colored reaction product at the site of the enzyme.

Step 105—removing unbound secondary antibody from the section of the biological sample, preferably via rinsing with water or other suitable rinsing medium.

Optional Step 106—performing enzyme substrate chromogen reaction, such as for counterstaining the tissue.

Step 107—mounting the section of the biological sample. In preferred embodiments, step 107 may comprise rapidly dehydrating the tissue and simultaneously embedding the tissue with application of a transparent alcohol and a water and alcohol soluble plastic. In further preferred embodiments, step 107 may comprise mounting the dehydrated tissue with a resin-based mounting medium.

Step 108—detecting secondary antibody bound to the section of the biological sample, preferably via observing the presence or absence of colored chromogen with a microscope or other visualization tool.

### ALTERNATIVE EMBODIMENTS OF INVENTION

An object of the present invention is to provide a method to stain tissues using immunohistochemistry such that the entire staining method can be completed in about 10 minutes. Other methods using immunohistochemistry have been described. However, these methods typically use standard immunohistochemical staining methods and reduce incubation times for each step in order to achieve rapid results. However, these methods have, for the most part, produced weak staining with low sensitivity. Such low sensitivity does not provide any added value over a conventional H&E stain, and these methods are rarely used in clinical practice.

For difficult cases, where either the diagnosis is uncertain or there are remaining questions regarding the surgical margins, a standard immunohistochemistry stain may be required. However, in these cases the stains are usually not completed until several days later. If additional finding are uncovered the patient may be recalled for additional surgery. The present invention of rapid immunohistochemical staining is able to overcome these limitations.

### Operation of Preferred Embodiment

The following Examples are shown to further illustrate the present Invention.

### Example 1. Tissue Preparation

Moh's micrographic surgery was used to obtain a skin biopsy containing a presumptive squamous cell carcinoma. The biopsy was frozen in an isopentane and dry ice bath. The frozen tissue was sectioned on a cryostat at −20 C at thickness of 6 micrometers (u), and attached to a pre-cooled silanized microscope slide. The slides with the attached tissues where then either dried at room temperature (air-dried) or sprayed with a cytological spray fixative composed of polyethylene glycol and ethanol and then dried at room temperature (fixed). Once dried the slides were either stained immediately or stored at −20 C prior to staining.

The purpose of the following experiment was to determine the best method for preparation and storage of the slides. Although storage of unstained slides is not a routine procedure in a clinical setting, for research purposes the ability to store unstained slides is advantageous in that it provides a consistent source of unstained materials over the course of these studies. Slides were stained by immunohistochemistry with the antibody to cytokeratin, clones AE1 and AE3 to demonstrate squamous cell carcinoma as follows:

1. AE1/AE3 primary antibody incubation for 20 minutes.

2. Anti-mouse HRP-Polymer incubation for 20 minutes.

3. DAB incubation for 5 minutes.

4. Counterstain with Hematoxylin for 3 minutes.

5. Mount and view with a microscope.

6. Brown staining due to DAB chromogen was scored on a semi-quantitative scale of 0 to 3 as follows:

A. 0=no staining.

B. 1=weak staining.

C. 2=moderate staining.

D. 3=strong staining.

TABLE 1
Results for Example 1. Tissue Preparation.

| Specimen | Scoring for | |
|---|---|---|
| Type | AE1/AE3 | Observations |
| Fresh air-dried | 2.0 | |
| Fresh Fixed | 2.5 | better morphology than air-dried |
| 3 day air-dried | 2.0 | |
| 7 day air-dried | 2.0 | |
| 21 day air-dried | 1.5 | |
| 3 day Fixed | 2.5 | better morphology than air-dried |
| 7 day Fixed | 2.5 | better morphology than air-dried |
| 21 day Fixed | 2.0 | better morphology than air-dried |

These results showed that both fresh and stored tissue samples were equivalent up to day 7. By day 21 there was only a slight decrease in staining. Furthermore, these studies showed that both air-dried and fixed tissue samples could be used. In general, the fixed tissue samples provided slightly stronger staining and improved morphology compared to their air-dried counterparts.

For the remainder of the Examples the following conditions were used unless otherwise indicated.

1. Microscope slides containing tissues that were spray fixed.

2. Microscope slides containing tissues that had been stored at −20 C for not greater than 7 days.

TABLE 2
Results of Permeabilization of Skin Biopsies.

| Permeabilization Conditions | | |
|---|---|---|
| Time | Temperature | Staining Results for AE1/AE3 |
| Not Done | Not Done | 2.0 |

| | | | |
|---|---|---|---|
| 1 | min | RT | 2.0 |
| 5 | min | RT | 2.0 |
| 30 | min | RT | 2.5 |
| 60 | min | RT | 3.0 |
| 1 | min | 90 C. | 3.0 |
| 5 | min | 90 C. | 3.0 |
| 30 | min | 90 C. | 3.0 |
| 60 | min | 90 C. | 3.0 |

Surprisingly we were able to achieve strong staining after only 1 minute incubation with the permeabilization reagent at 90 C. Having already achieved our goal of increasing staining, while only adding 1 minute to the overall protocol time, we did not test additional variables.

### Example 3. Fc-Specific Secondary Antibody

The following secondary antibodies were tested:

1. Goat anti-mouse IgG (heavy and light chain specific) polymer with horseradish peroxidase (GAM-HRP polymer).

2. Goat anti-mouse IgG (Fc-specific) polymer with horseradish peroxidase (GAM-Fc-HRP polymer).

Slides with tissues were prepared and permeabilized according to Examples 1 and 2. Slides were stained according to Example 1 for AE1/AE3 except the GAM-Fc-HRP polymer was compared to the GAM-HRP polymer as follows:

1. AE1/AE3 primary antibody incubated for 20 minutes.

2. HRP polymer secondary antibody incubated for 20 minutes.

3. DAB chromogen incubated for 5 minutes.

4. Counterstained with Hematoxylin for 3 minutes.

5. Mounted and observed under the microscope.

6. Graded on a scale of 0 to 3.

TABLE 3
Results of Test of GAM-Fc-HRP polymer vs. GAM-
HRP polymer using sequential incubation.

| Secondary Antibody | Staining results for AE1/AE3 |
|---|---|
| GAM-HRP Polymer | 3.0 |
| GAM-Fc-HRP | 3.0 |
| Polymer | |

These results showed that both the GAM-HRP polymer and the GAM-Fc-HRP polymer performed comparably using a sequential incubation.

### Example 4. Test of GAM-Fc-HRP Polymer Vs. GAM-HRP Polymer Using Simultaneous Incubation

The following three conditions were tested:

1. Pre-mix the primary antibody and the secondary polymer, and then apply to the tissue as a single step.

2. Apply the primary antibody onto the slide first and then apply the secondary polymer onto the tissue so that both reagents incubate simultaneously.

3. Apply the primary antibody onto the slide first, wait for a period of time and drain off excess primary antibody without rinsing, and then add the secondary polymer onto the tissue.

TABLE 4
Results of Test of various incubation
methods for GAM-Fc-HRP polymer.

| Secondary | Test | Staining results |
|---|---|---|
| Antibody | Conditions | for AE1/AE3 |
| GAM-HRP | Pre-mix with primary antibody | 0.0 |
| GAM-HRP | Apply primary antibody first | 0.5 |
| | Then apply secondary antibody | |
| GAM-HRP | Apply primary antibody first | 1.0 |
| | Drain Slide | |
| | Apply secondary antibody | |
| GAM-Fc-HRP | Pre-mix with primary antibody | 2.0 |
| GAM-Fc-HRP | Apply primary antibody first | 2.5 |
| | Then apply secondary antibody | |
| GAM-Fc-HRP | Apply primary antibody first | 3.0 |
| | Drain Slide | |
| | Apply secondary antibody | |

These results show that the GAM-Fc-HRP polymer reagent gave significantly stronger staining compared to the GAM-HRP polymer when the polymer reagent was allowed to react in the presence of the primary antibody.

### Example 5. Rapid Dehydration and Mounting

A dehydrating reagent was prepared by preparing a solution of 10% polyvinyl in ethanol. After immunohistochemical staining and Hematoxylin counterstaining the slides were prepared for dehydration and mounting as follows:

Standard method of dehydration.

1. Bath of 50% water and 50% alcohol for 3 min.

2. Bath of 25% water and 75% alcohol for 3 min.

3. Bath of 0% water and 100% alcohol for 3 min.

4. Bath of 50% alcohol and 50% xylene for 3 min.

5. Bath of 25% alcohol and 75% xylene for 3 min.

6. Bath of 0% alcohol and 100% xylene for 3 min.

7. One drop of resin-mounting medium in xylene.

8. Glass coverslip.

Rapid dehydration method (method 400).

1. Bath of dehydration reagent, quick dunk, 1-2 seconds.

2. Remove slides, drain, and air-dry, 10 seconds.

3. One drop of resin-mounting medium in xylene.

4. Glass coverslip.

TABLE 5
Results of Comparison of Rapid dehydration
to standard dehydration.

| Method | Staining | Optical Resolution | Morphology |
|---|---|---|---|
| Standard | 3.0 | Excellent | Excellent. |
| Rapid | 3.0 | Excellent. | Excellent |

These results show that the rapid dehydration method (method 400) was equivalent to the standard dehydration method in terms of staining, optical resolution, and morphology. However, the rapid dehydration method took less than 20 seconds to complete compared to about 20 minutes for the standard dehydration method.

### Example 6. Rapid Immunohistochemistry

Having completed all of the preliminary steps to develop a rapid immunohistochemistry staining method 100, the following protocol was tested to see whether the individual incubation times could be reduced with a goal of achieving a complete immunohistochemical stain within 10 minutes.

1. Permeabilization reagent was pre-heated in a microwave for 1 minute or until a temperature of approximately 90 C was reached. This step was performed while the slides were simultaneously being prepared, thus adding no additional time to the protocol.

2. Slides were placed into the pre-heated permeabilization reagent for 1 minute. This step was performed on a bench top at room temperature, while the permeabilization reagent cooled slightly.

3. Slides were transferred briefly to a buffer bath.

4. Slides were arranged horizontally and 100 ul of primary antibody was overlaid the tissue and incubated for 2.5 minutes.

5. Excess antibody was drained from the slide, and without rinsing 100 ul of GAM-Fc-HRP Polymer or GAR-Fc-HRP Polymer for mouse or rabbit primary antibodies respectively was overlaid the tissue and incubated for 2.5 minutes.

6. Slides were rinsed to remove excess primary antibody and polymer, then the tissues were overlaid with 100 ul DAB and incubated for 2.5 minutes.

7. Slides were rinsed in water and then counterstained with Hematoxylin for 10 seconds.

8. Slides were rinsed in water and then dehydrated with rapid dehydration reagent for 20 seconds.

9. Slides were mounted with mounting medium and coverslipped.

10. Total time of incubations was 9 minutes. Additional time for rinsing slides added approximately 1 additional minute for a total run time of about 10 minutes.

Table 6 shows a comparison of staining results comparing Rapid Immunohistochemistry of Example 6 to Standard Immunohistochemistry of Example 1.

TABLE 6
Comparison of Rapid Immunohistochemistry
to Standard Immunohistochemistry.

| | | | Staining Results | |
|---|---|---|---|---|
| | | | Immunohistochemistry | |
| Primary | Primary | Secondary | Method | |
| Antibody | Species | Antibody | Rapid | Standard |
| AE1/AE3 | Mouse | GAM-Fc-HRP Polymer | 3.0 | 3.0 |
| Ki67 | Rabbit | GAR-Fc-HRP Polymer | 3.0 | 3.0 |
| Vimentin | Rabbit | GAR-Fc-HRP Polymer | 3.0 | 3.0 |
| Melanoma | Mouse | GAM-Fc-HRP Polymer | 3.0 | 3.0 |
| (HMB45) | | | | |

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A method for rapid immunohistochemical detection of an antigen from a biological sample, the method comprising the steps of:
depositing a section of the biological sample on a slide;
permeabilizing the section of the biological sample;
incubating the section of the biological sample with a secondary antibody having a detectable label;
removing unbound secondary antibody from the section of the biological sample;
mounting the section of the biological sample; and
detecting secondary antibody bound to the section of the biological sample.

2. The method of claim 1, wherein the section of the biological sample is produced by freezing the biological sample and then sectioning the biological sample.

3. The method of claim 1, wherein the section of the biological sample is produced by embedding the biological sample in paraffin and then sectioning the biological sample.

4. The method of claim 1, wherein the section of the biological sample is permeabilized with a permeabilization reagent that is heated to between 25 degrees and 95 degrees Celsius.

5. The method of claim 1, wherein the secondary antibody is specific to a Fc portion of a primary antibody also applied to the section of the biological sample.

6. The method of claim 1, wherein the section of the biological sample is incubated with the secondary antibody after incubating the section of the biological sample with a primary antibody that is specific to the antigen, and wherein the secondary antibody is specific to the Fc portion of the primary antibody.

7. The method of claim 1, wherein the section of the biological sample is concurrently incubated with the secondary antibody and with a primary antibody that is specific to the antigen, and wherein the secondary antibody is specific to the Fc portion of the primary antibody.

8. The method of claim 1, wherein the detectable label comprises an enzyme molecule.

9. The method of claim 1, wherein the detectable label comprises a polymer having two or more enzyme molecules bound to it.

10. The method of claim 1, wherein the detectable label comprises a fluorescent tag.

11. The method of claim 1, wherein the step of mounting the section of the biological sample comprises applying a mixture of an alcohol and an alcohol and water soluble polymer to the section of the biological sample.

12. The method of claim 11, wherein a resin-based mounting medium is applied to the section of the biological sample after the alcohol has dried.

13. A method for rapid immunohistochemical detection of an antigen from a biological sample during a cancer removal procedure, the method comprising the steps of:
depositing a section of the biological sample on a slide, wherein the section of the biological sample is produced by freezing the biological sample and then sectioning the biological sample;
permeabilizing the section of the biological sample;
incubating the section of the biological sample with a secondary antibody having a detectable label, wherein the secondary antibody is specific to a Fc portion of a primary antibody also applied to the section of the biological sample;
removing unbound secondary antibody from the section of the biological sample;
mounting the section of the biological sample, wherein the step of mounting the section of the biological sample comprises applying a mixture of an alcohol and an alcohol and water soluble polymer to the section of the biological sample; and
detecting secondary antibody bound to the section of the biological sample.

14. The method of claim 13, wherein the section of the biological sample is permeabilized with a permeabilization reagent that is heated to between 25 degrees and 95 degrees Celsius.

15. The method of claim 13, wherein the section of the biological sample is incubated with the secondary antibody after incubating the section of the biological sample with a primary antibody that is specific to the antigen, and wherein the secondary antibody is specific to the Fc portion of the primary antibody.

16. The method of claim 13, wherein the section of the biological sample is concurrently incubated with the secondary antibody and with a primary antibody that is specific to the antigen, and wherein the secondary antibody is specific to the Fc portion of the primary antibody.

17. The method of claim 13, wherein the detectable label comprises an enzyme molecule.

18. The method of claim 13, wherein the detectable label comprises a polymer having two or more enzyme molecules bound to it.

19. The method of claim 13, wherein the detectable label comprises a fluorescent tag.

20. The method of claim 13, wherein a resin-based mounting medium is applied to the section of the biological sample after the alcohol has dried.
